(19) United States
(12) Patent Application Publication
(10) Pub. No.: US 2011/0082428 A1
(43) Pub. Date: Apr. 7, 2011

(54) Safety structure for covering syringe needle

(76) Inventor: Wu-Shun Huang, Taipei (TW)

(21) Appl. No.: 12/588,187

(22) Filed: Oct. 7, 2009

(51) Int. Cl. A61M 5/32

(52) U.S. Cl. 604/198

(57) ABSTRACT

A safety structure for covering a syringe needle includes a safety sleeve and a hub provided with a sideward projection. The safety sleeve is fitted around the hub and provided with an axial sliding slot, a locating slot laterally extended from the sliding slot, a locating hole located at a bottom of the locating slot behind a locating section, and two opposing protrusions located in the locating slot between the sliding slot and the locating section. When the projection is abutted on a bottom of the sliding slot, the needle is exposed from the safety sleeve; when the projection is reversibly located behind the two opposing protrusions, the needle is temporarily enclosed in the safety sleeve and protected against contamination; and when the projection is moved behind the locating section into the locating hole, the needle is permanently enclosed in the safety sleeve without the risk of stabbing a user.

### FIELD OF THE INVENTION

The present invention relates to a safety structure for covering syringe needle, and more particularly to a safety sleeve being adjustably fitted around a needle hub of a syringe to enclose a syringe needle therein and thereby protecting a user picking up the syringe for injection against unexpected stabbing by the needle, making the syringe safer for use.

### BACKGROUND OF THE INVENTION

A syringe for medical purpose usually includes a protective cap covering a needle of the syringe. Before the use of the syringe, the protective cap is always closed onto the hub to enclose the needle therein to serve as a safety guard. Once the syringe has been used, the protective cap removed from the needle is not allowed to put back to the needle for security protection. That is, to prevent unexpected stabbing by the exposed and used needle and possible infection caused by such stabbing, the conventional cap is designed for use once and should not be reused. However, in practice, to use the syringe to inject a patient, a nursing person at a nursing station would first remove the protective cap from the needle for drawing a desired injection fluid into the syringe, and then send the prepared syringe to the ward for injecting the patient. During this process, to avoid the risk of being stabbed by the needle when trying to replace the protective cap on the needle, or because the protective cap is allowed for use one time only, the protective cap removed from the needle is put aside without being used to cover the needle again, so that the needle is kept exposed to the open environment. Under this condition, it is very possible someone nearby the syringe is unexpectedly stabbed by the exposed needle, and the exposed needle is also subject to contamination by the external environment. After injection, the previously removed protective cap is replaced onto the hub to cover the used needle, or a disposable protective cap is used to cover the used needle, lest someone else should be stabbed by the used needle. Therefore, the conventional protective cap for syringe needle requires troublesome handling procedures and the user is still subject to the risk of being stabbed by the needle during different stages of handling the syringe. It is therefore tried by the inventor to develop a safety structure for covering syringe needle, so as to protect users, such as nurses or other nursing persons, against unexpected stabbing by the syringe needle when picking up and handling the prepared syringe or disposing the used syringe, and to prevent the needle from being contaminated by the external environment during the above procedures.

### SUMMARY OF THE INVENTION

A primary object of the present invention is to provide a safety structure for covering syringe needle, with which a syringe needle can be temporarily enclosed in a safety sleeve before being used to inject and permanently enclosed in the safety sleeve after having been used to inject, so that a user picking up, handling or disposing the syringe is protected against unexpected stabbing by the needle, and the syringe is safer for use.

To achieve the above and other objects, the safety structure for covering syringe needle according to an embodiment of the present invention includes a safety sleeve and a needle hub of a syringe provided with a sideward projection. The safety sleeve is provided at a rear open end with a cutout, and on a wall with a sliding slot axially forward extended from the cutout by a predetermined length, a stop section located in the sliding slot at a predetermined point, a locating slot laterally extended from one side of the sliding slot by a predetermined length to locate in front of the stop section, a locating hole located at a bottom of the locating slot, and a locating section located at a junction between the locating hole and the locating slot.

When the safety sleeve is fitted around a front portion of the syringe with the projection on the needle hub aligned with the cutout, the projection can be moved through the stop section into the sliding slot to finally abut on a bottom of the sliding slot for a needle on the hub to expose from a front open end of the safety sleeve; and when the syringe has been used to draw an injection fluid or has been used to inject, the safety sleeve can be slid for the projection on the hub to move along the sliding slot to the locating slot, and be further moved through the locating section into the locating hole. At this point, the projection is permanently held in the locating hole for the safety sleeve to permanently enclose the whole needle therein, protecting a user picking up, handling or disposing the syringe against unexpected stabbing by the needle, and making the syringe safer for use.

In the safety structure for covering syringe needle according to the present invention, two opposing protrusion are further provided in the locating slot between the sliding slot and the locating section. When the safety sleeve is moved for the projection to pass through the two opposing protrusions, the safety sleeve is located at a position on the syringe capable of enclosing the whole needle therein. The projection can also be moved reversely to pass through the two opposing protrusions and return to the bottom of the sliding slot, so that the needle is exposed from the safety sleeve for use. Therefore, when the needle has been used to draw an injection fluid but has not been used to inject, the needle can be temporarily enclosed in the safety sleeve by moving the projection to locate it behind the two opposing protrusions, lest the needle should be contaminated by the external environment or unexpectedly stabbing a user.

In the safety structure for covering syringe needle according to the present invention, the stop section in the sliding slot includes two substantially triangular lugs separately located at two opposite sides of the sliding slot to extend into the sliding slot by a predetermined distance. Each of the two triangular lugs has a slant rear edge to allow the projection to smoothly pass through the stop section into the sliding slot, and a horizontal and straight front edge to prevent the projection from moving backward out of the cutout.

In the safety structure for covering syringe needle according to the present invention, the locating section in the locating slot includes two substantially triangular lugs separately located at two opposite sides of the locating hole to extend into the locating hole by a predetermined distance. One edge of each of the two lugs facing away from the locating hole is a slant edge to allow the projection to smoothly pass through the locating section into the locating hole, and another edge of each of the two lugs facing toward the locating hole is a straight edge to prevent the projection from moving backward out of the locating hole.

In the safety structure for covering syringe needle according to the present invention, a first and a second axial slit are further provided on the safety sleeve closely adjacent to one of the two lugs of the stop section and the locating section, respectively, giving those lugs a certain degree of elasticity, so that the projection on the hub can move through the stop section and the locating section more easily.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is an exploded perspective view showing a safety structure for covering syringe needle according to the present invention;

FIG. 2 is a perspective view of a first embodiment of a safety sleeve adopted in the present invention;

FIG. 3 is an assembled view of FIG. 1 with a syringe needle exposed from a front open end of the safety sleeve for use;

FIGS. 4A and 4B are fragmentary perspective views showing the manner of holding the safety sleeve to a position for temporarily enclosing the whole syringe needle therein;

FIG. 5 is an assembled perspective view of FIG. 1 showing the safety sleeve is moved to a position for permanently enclosing the whole syringe needle therein;

FIG. 6 is a perspective view showing a second embodiment of the safety sleeve for the present invention;

FIG. 7 is a perspective view showing a third embodiment of the safety sleeve for the present invention;

FIG. 8A is a perspective view showing a fourth embodiment of the safety sleeve for the present invention; and

FIG. 8B is a fragmentary sectional view showing the engagement of the safety sleeve of FIG. 8A with a syringe to cover a syringe needle.

### DETAILED DESCRIPTION OF THE PREFERRED EMBODIMENTS

The present invention will now be described with some preferred embodiments thereof. For the purpose of easy to understand, elements that are the same in the preferred embodiments are denoted by the same reference numerals.

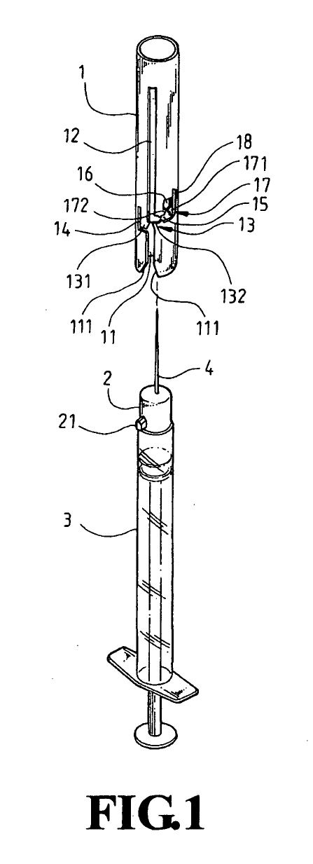

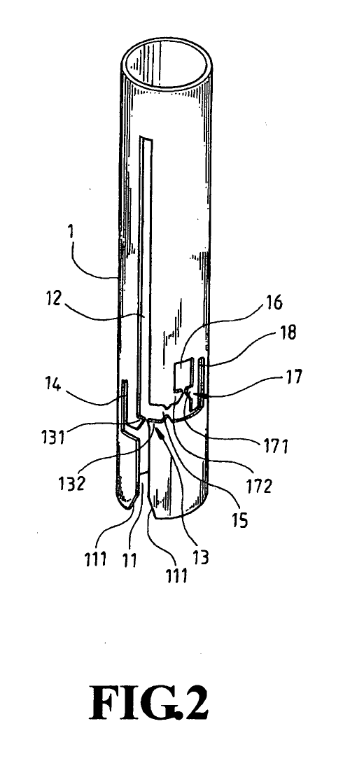

Please refer to FIG. 1 that is an exploded perspective view showing a safety structure for covering syringe needle according to the present invention. As shown, the safety structure includes a safety sleeve 1 and a needle hub 2 provided at a predetermined position with a radially outward projection 21. Please also refer to FIG. 2 that is a perspective view of the safety sleeve 1 according to a first embodiment thereof. As shown, the safety sleeve 1 in the first embodiment is provided at a rear open end with a cutout 11, a rearmost end of which is expanded to provide two beveled lateral edges 111, and a front end of which is axially extended forward by a predetermined length to form a longitudinal sliding slot 12 on the safety sleeve 1. A stop section 13 is provided in the sliding slot 12 at a predetermined point. The stop section 13 includes two substantially triangular lugs 131, 132, which are located at two opposite sides of the sliding slot 12 to extend into the sliding slot 12 by a predetermined distance. The lugs 131, 132 each have a slant rear edge to allow the projection 21 to smoothly pass through the stop section 13 into the sliding slot 12, and a horizontal and straight front edge to prevent the projection 21 from moving backward out of the cutout 11. A first axial slit 14 is formed on the safety sleeve 1 closely adjacent to the lug 131 and communicable with the sliding slot 12 to thereby give the lug 131 a certain degree of elasticity.

A transverse locating slot 15 is formed on the safety sleeve 1 to laterally extend from one side of the sliding slot 12 by a predetermined length and locate in front of the lug 132 of the stop section 13. And, a bottom of the locating slot 15 distal from the sliding slot 12 is further turned forward to provide a locating hole 16. A locating section 17 is provided at a junction between the locating slot 15 and the locating hole 16, and includes two substantially triangular lugs 171, 172 located at two opposite sides of the locating hole 16 to extend into the locating hole 16 by a predetermined distance. One edge of each of the triangular lugs 171, 172 facing away from the locating hole 16 is a slant edge to allow the projection 21 to smoothly pass through the locating section 17 into the locating hole 16, and another edge of each of the triangular lugs 171, 172 facing toward the locating hole 16 is a straight edge to prevent the projection 21 from moving backward out of the locating hole 16. A second axial slit 18 is formed on the safety sleeve 1 closely adjacent to the lug 171 and communicable with the locating slot 15 to thereby give the lug 171 a certain degree of elasticity.

Two opposing protrusions 121 are further provided in the locating slot 15 between the sliding slot 12 and the locating section 17, such that the projection 21 is allowed to reversibly pass through the two opposing protrusions 121. That is, the projection 21 can be temporarily located behind the two opposing protrusions 121.

As can be seen from FIG. 1, the needle hub 2 is mounted to a front end of a syringe 3 for a needle 4 to fixedly set thereto. The needle hub 2 is provided on a peripheral wall at a predetermined position with the radially outward projection 21. A front side of the projection 21 facing toward a front end of the needle hub 2 is forward tapered. Meanwhile, one side of the projection 21 for facing toward the locating hole 16, which can be oriented upward, downward or sideward in different embodiments, is tapered, and an opposing side of the projection 21 is a flat side. Therefore, the tapered side of the projection 21 allows the projection 21 to smoothly pass through the locating section 17 into the locating hole 16, and the flat side of the projection 21 prevents the projection 21 from moving backward out of the locating hole 16.

With the above arrangements, the safety sleeve 1 can be fitted around a front portion of the syringe 3, such that the projection 21 on the needle hub 2 is aligned with the cutout 11 and moved through the stop section 13 into the sliding slot 12 to finally abut on a bottom of the sliding slot 12. At this point, the needle 4 is exposed from a front open end of the safety sleeve 1. Since the cutout 11 has an expanded rearmost end to provide two opposing beveled lateral edges 111, the projection 21 can be easily moved into the cutout 11 and accordingly, the sliding slot 12. Once the projection 21 has been moved into the sliding slot 12, the projection 21 is prevented by the horizontal and straight front edge of the stop section 13 from moving backward to pass through the stop section 13. After an injection fluid has been drawn into the syringe 3 and before the syringe 3 is used to inject, a user can move the safety sleeve 1 for the projection 21 to slide along the sliding slot 12 and the locating slot 15 to pass through the two opposing protrusions 121. At this point, the safety sleeve 1 is moved forward to enclose the needle 4 therein, and the projection 21 is held behind the two opposing protrusions 121 to temporarily hold the safety sleeve 1 in the position capable of enclosing the whole needle 4 therein. With the needle 4 enclosed in the safety sleeve 1, a nursing person is protected against unexpected stabbing by the needle 4 when trying to pick up the syringe 3, and the needle 4 is protected against the risk of being contaminated. To use the prepared syringe 3 to inject a patient, the safety sleeve 1 is moved again for the projection 21 to move reversely and pass through the two opposing protrusion 121 into the sliding slot 12 to abut on the bottom of the sliding slot 12. At this point, the needle 4 is exposed from the safety sleeve 1 for injection. After the syringe 3 has been used to inject, the safety sleeve 1 can be moved for the projection 21 to move along the sliding slot 12 to the locating slot 15 into the locating hole 16 via the locating section 17. At this point, the projection 21 is located in the locating hole 16 with the flat side of the projection 21 abutted on the straight edges of the two lugs 171, 172 of the locating section 17, and is therefore prevented from moving reversely out of the locating hole 16. As a result, the projection 21 is permanently held in the locating hole 16 to thereby permanently hold the safety sleeve 1 to the position capable of enclosing the whole needle 4 therein. Therefore, the used needle 4 could not stab anyone to cause infection, and the syringe 3 is safer for use.

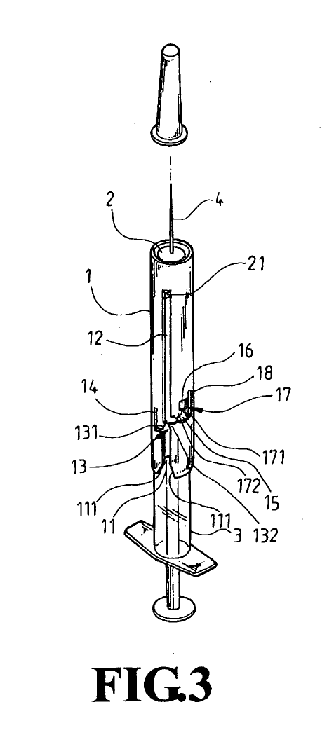

Please refer to FIG. 3 that shows the safety sleeve 1 fitted around the syringe 3 is located at a position for the needle 4 to expose from the front open end of the safety sleeve 1. As shown, to use the safety structure for covering syringe needle, the safety sleeve 1 is fitted around the front portion of the syringe 3, such that the projection 21 on the needle hub 2 is aligned with the cutout 11 and moved to the stop section 13. With the first axial slit 14 provided on the safety sleeve 1 closely adjacent to the lug 131 of the stop section 13 to provide the lug 131 with a certain degree of elasticity, and with the tapered front side of the projection 21, the projection 21 is allowed to successfully move through the two substantially triangular lugs 131, 132 of the stop section 13 into the sliding slot 12 to finally abut on the bottom of the sliding slot 12. At this point, the needle 4 is exposed from the front open end of the safety sleeve 1. On the other hand, with the flat side of the projection 21 facing toward the horizontal and straight edges of the two lugs 131, 132 of the stop section 13, the projection 21 is prevented by the stop section 13 from moving rearward out of the cutout 11.

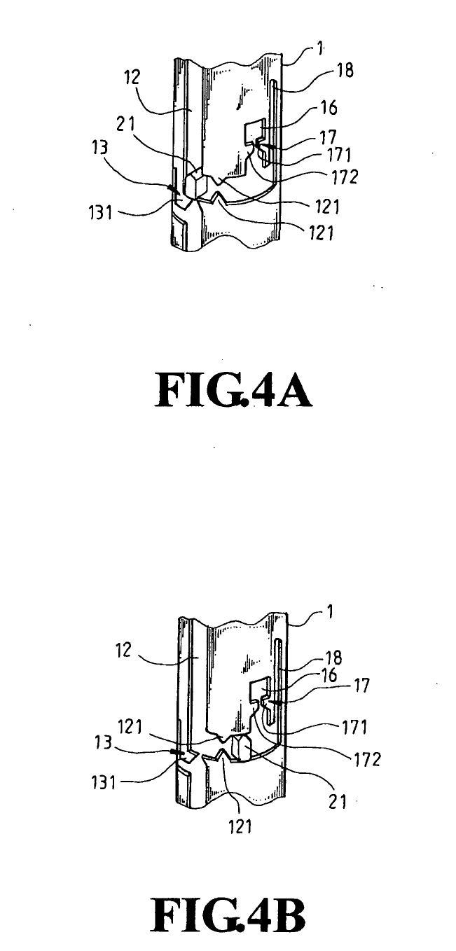

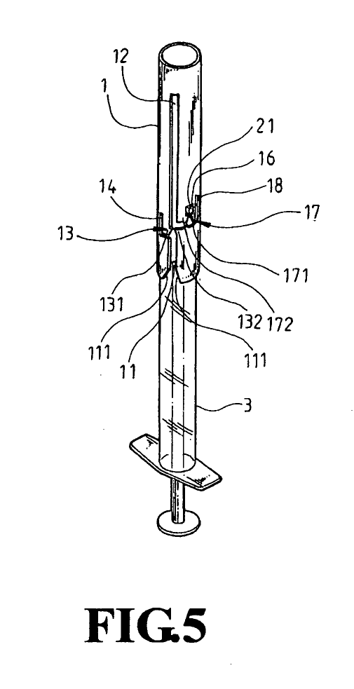

FIGS. 4A and 4B are fragmentary perspective views showing the manner of holding the safety sleeve 1 to a position for temporarily enclosing the whole syringe needle 4 therein, and FIG. 5 is an assembled perspective view of FIG. 1 showing the safety sleeve 1 is moved to a position for permanently enclosing the whole syringe needle 4 therein As shown, when an injection fluid has been drawn into the syringe 3 via the needle 4, the safety sleeve 1 can be slid forward to enclose the whole needle 4, and meanwhile, the projection 21 is brought to slide rearward along the sliding slot 12. When the projection 21 reaches at the stop section 13, the projection 21 can be further moved into the locating slot 15 to reversibly pass through the two opposing protrusions 121 and locate in front of the locating section 17. With the projection 21 located at this position, the safety sleeve 1 is temporarily held to the position capable of enclosing the whole needle 4 therein until the syringe 3 is used to inject a patient. After the syringe 3 has been used to inject, the projection 21 can be moved along the locating slot 15 to the locating section 17. With the two substantially triangular lugs 171, 172 of the locating section 17 located at two opposite sides of the locating hole 16, and with the second axial slit 18 provided on the safety sleeve 1 closely adjacent to the lug 171 of the locating section 17 to provide the lug 171 with a certain degree of elasticity, the projection 21 can be further moved through the locating section 17 into the locating hole 16. With the flat side of the projection 21 abutted on the straight edges of the two lugs 171, 172 of the locating section 17, the projection 21 is permanently held in the locating hole 16 without the risk of moving reversely out of the locating hole 16. As a result, the safety sleeve 1 is permanently held to the position of enclosing the whole needle 4 therein, and the needle 4 would not stab anyone, making the syringe 3 safer for use.

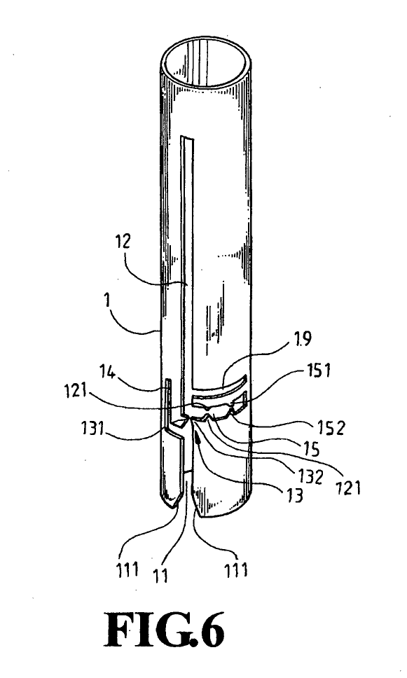

FIG. 6 is a perspective view of the safety sleeve 1 according to a second embodiment thereof. As shown, the safety sleeve 1 in the second embodiment is generally structurally similar to the first embodiment, except that the locating hole 16 and the locating section 17 are omitted, the locating slot 15 is internally provided near the bottom thereof with two substantially triangular lugs 151, 152, which are located at two opposite sides of the locating slot 15 to extend into the locating slot 15 by a predetermined distance, and a transverse slit 18 is laterally extended from one side of the sliding slot 12 to closely locate in front of the locating slot 15 and thereby gives the lug 151 of the locating slot 15 a certain degree of elasticity. With these arrangements, the projection 21 on the needle hub 2 is allowed to successfully pass through the lugs 121 and be temporarily held in the locating slot 15 behind the two lugs 121.

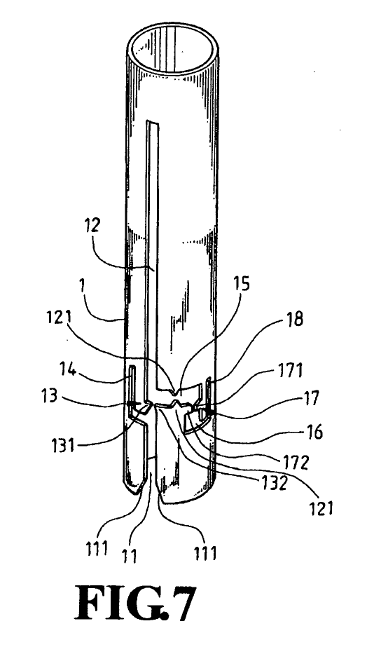

FIG. 7 is a perspective view of the safety sleeve 1 according to a third embodiment thereof. As shown, the safety sleeve 1 in the third embodiment is generally structurally similar to the first embodiment, except that the bottom of the transverse locating slot 15 is further turned backward to form the locating hole 16. The locating section 17 is still located at the junction between the locating slot 15 and the locating hole 16, and the second axial slit 18 is located closely adjacent to the lug 171. Again, the projection 21 is allowed to successfully pass through the lugs 121 and be temporarily held in the locating slot 15 behind the two lugs 121. Or, the projection 21 having been moved into the locating slot 15 can successfully move through the locating section 17 into the locating hole 16 and be permanently held in the locating hole 16 by the locating section 17.

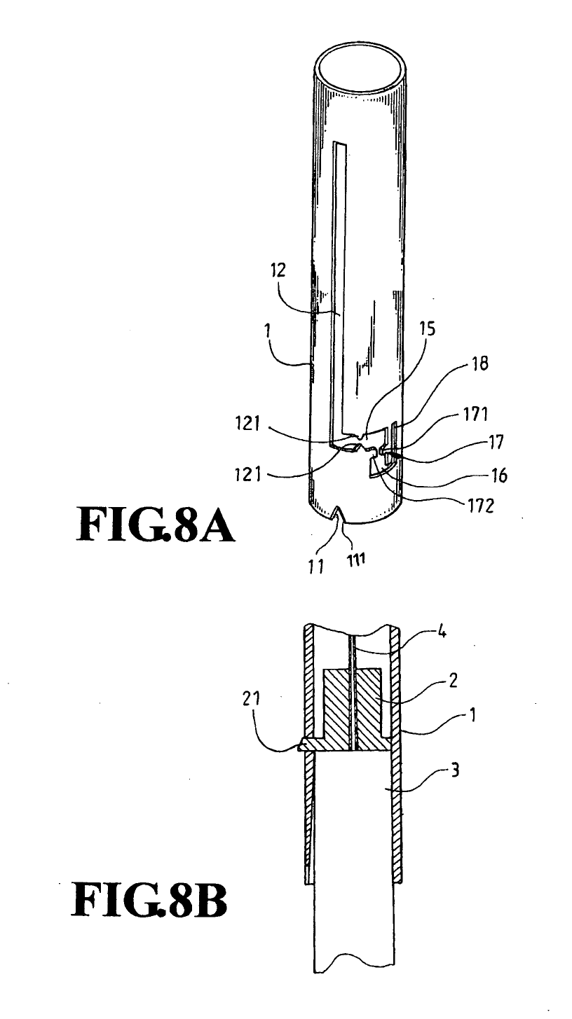

Please refer to FIG. 8A that is a perspective view showing a fourth embodiment of the safety sleeve 1, and to FIG. 8B that is a fragmentary sectional view showing the engagement of the safety sleeve of FIG. 8A with the syringe 3 to cover the needle 4 therein. As shown, the safety sleeve 1 in the fourth embodiment is generally structurally similar to the safety sleeve 1 in the previous embodiments, except that the stop section 13 and a length of the sliding slot 12 behind the locating slot 15 are omitted from the safety sleeve 1, and a predetermined portion on an inner wall surface of the safety sleeve 1 is gradually thinned from behind the sliding slot 12 and the locating slot 15 toward the beveled lateral edges 111 of the cutout 11. Meanwhile, the projection 21 on the needle hub 2 has a radially outward beveled front face, so that it has a narrower front portion and wider rear portion. To use the safety structure of the present invention, the safety sleeve 1 is fitted around the needle hub 2 from the front end of the syringe 3 with the projection 21 aligned with the gradually rearward thinned inner wall surface of the safety sleeve 1, such that the projection 21 with the beveled face is allowed to slide forward along the gradually rearward thinned inner wall surface of the safety sleeve 1 into the sliding slot 12. The safety sleeve 1 can be moved for the projection 21 to slide along the sliding slot 12 and the locating slot 15 to locate behind the two opposing protrusions 121 or the locating section 17. When the projection 21 is located behind the two opposing protrusions 121, the safety sleeve 1 is temporarily held to the position for enclosing the whole needle 4 therein; and when the projection 21 is located behind the locating section 17, the safety sleeve 1 is permanently held to the position for enclosing the whole needle 4 therein. In the case of locating the projection 21 behind the locating section 17, with the two substantially triangular lugs 171, 172 of the locating section 17 located at two opposite sides of the locating hole 16, and with the axial slit 18 provided on the safety sleeve 1 closely adjacent to the lug 171 of the locating section 17 to provide the lug 171 with a certain degree of elasticity, the projection 21 can be smoothly moved through the locating section 17 into the locating hole 16, and be permanently held in the locating hole 16 by the locating section 17.

In brief, the safety structure for covering syringe needle according to the present invention includes a safety sleeve provided with a sliding slot, a stop section, a locating slot, a locating section and a locating hole, and a needle hub with a projection that cooperates with the safety sleeve to completely cover a needle of a syringe in the safety sleeve when the syringe has been used to draw in an injection fluid or has been used to inject a patient, so that the needle is protected against contamination and the nursing person is protected against stabbing by the needle. Therefore, the present invention is a practical design making the syringe safer for use.

The present invention has been described with some preferred embodiments thereof and it is understood that the first and the locating section can be differently configured and many other changes and modifications in the described embodiments can be carried out without departing from the scope and the spirit of the invention that is intended to be limited only by the appended claims.

What is claimed is:

1. A safety structure for covering a syringe needle, comprising:
a safety sleeve being provided at a rear open end with a cutout, and on a wall with a sliding slot axially forward extended from a front end of the cutout by a predetermined length, a stop section located in the sliding slot at a predetermined point, a locating slot laterally extended from one side of the sliding slot by a predetermined length and located in front of the stop section, a locating hole extended from a bottom of the locating slot, and a locating section provided at a junction between the locating slot and the locating hole; and
a needle hub being mounted to a front end of a syringe for a needle to fixedly set thereto, and being provided on a peripheral wall at a predetermined position with a radially outward projection;
whereby when the safety sleeve is fitted around a front portion of the syringe with the projection on the needle hub aligned with the cutout, the projection is moved through the stop section into the sliding slot to finally abut on a bottom of the sliding slot for the needle to expose from a front open end of the safety sleeve; and when the syringe has been used to draw an injection fluid or has been used to inject, the safety sleeve can be slid forward for the projection to move in the sliding slot; and when the projection is moved backward along the sliding slot to the locating slot and is further moved through the locating section into the locating hole, the projection is permanently held in the locating hole by the locating section without the risk of moving reversely out of the locating hole, and the safety sleeve is permanently maintained at the forward position to enclose the whole needle therein, preventing the used needle from unexpectedly stabbing a user and making the syringe safer for use.

2. The safety structure for covering a syringe needle as claimed in claim 1, wherein the stop section includes two lugs separately located at two opposite sides of the sliding slot to extend into the sliding slot by a predetermined distance; and each of the two lugs having a slant rear edge and a horizontal and straight front edge.

3. The safety structure for covering a syringe needle as claimed in claim 2, further comprising a first axial slit formed on the safety sleeve to locate closely adjacent to one of the two lugs of the stop section and communicate with the sliding slot to thereby give that lug a certain degree of elasticity.

4. The safety structure for covering a syringe needle as claimed in claim 1, wherein the locating section includes two lugs separately located at two opposite sides of the locating hole to extend into the locating hole by a predetermined distance; and each of the two lugs having a slant edge facing away from the locating hole and a horizontal and straight edge facing toward to locating hole.

5. The safety structure for covering a syringe needle as claimed in claim 4, further comprising a second axial slit formed on the safety sleeve to locate closely adjacent to one of the two lugs of the locating section and communicate with the locating slot to thereby give that lug a certain degree of elasticity.

6. The safety structure for covering a syringe needle as claimed in claim 1, wherein a rearmost end of the cutout is expanded to provide two beveled lateral edges, allowing the projection to be easily aligned with and moved into the cutout.

7. The safety structure for covering a syringe needle as claimed in claim 1, wherein one side of the projection on the needle hub facing toward a front end of the needle hub is forward tapered to allow the projection to smooth pass through the stop section on the safety sleeve.

8. The safety structure for covering a syringe needle as claimed in claim 1, further comprising two opposing protrusions provided in the locating slot between the sliding slot and the locating section; the projection on the hub being allowed to reversibly pass through the two opposing protrusions; whereby when the projection is forward moved through the two opposing protrusions to locate behind them, the safety sleeve is temporarily held to a position capable of enclosing the whole needle therein, so that the needle that has been used to draw an injection fluid but has not been used to inject is prevented from being contaminated by external environment and unexpectedly stabbing a user.

9. The safety structure for covering a syringe needle as claimed in claim 1, wherein one face of the projection on the hub facing toward the locating hole is tapered to allow the projection to smooth pass through the locating section on the safety sleeve.

10. A safety structure for covering a syringe needle, comprising:
a safety sleeve being provided on a rear open end with a cutout, on a wall at a predetermined position with a sliding slot axially extended by a predetermined length, a locating slot laterally extended from a predetermined point of the sliding slot by a predetermined length, a locating hole extended from a bottom of the locating slot distal from the sliding slot, and a locating section located at a junction between the locating slot and the locating hole; and a predetermined portion on an inner wall surface of the safety sleeve being gradually thinned from behind the sliding slot and the locating slot toward the cutout at the rear open end of the safety sleeve; and
a needle hub being mounted to a front end of a syringe for a needle to fixedly set thereto, and being provided on a peripheral wall at a predetermined position with a radially outward projection, and the projection having a radially outward beveled front face;
whereby when the safety sleeve is fitted around a front portion of the syringe from the front end thereof, the projection with the radially outward beveled front face is allowed to slide from the cutout at the rear open end of the safety sleeve along the gradually rearward thinned inner wall surface into the sliding slot to finally abut on a bottom of the sliding slot for the needle to expose from a front open end of the safety sleeve; and when the syringe has been used to draw an injection fluid or has been used to inject, the projection is moved rearward in the sliding slot to the locating slot and further moved through the locating section into the locating hole and be permanently held in the locating hole without the risk of moving reversely out of the locating hole, so that the safety sleeve is permanently maintained at the forward position to enclose the whole needle therein, preventing the used needle from unexpectedly stabbing a user and making the syringe safer for use.

11. The safety structure for covering a syringe needle as claimed in claim 10, wherein the locating section includes two lugs separately located at two opposite sides of the locating hole to extend into the locating hole by a predetermined distance; and each of the two lugs having a slant rear edge and a horizontal and straight front edge.

12. The safety structure for covering a syringe needle as claimed in claim 10, further comprising an axial slit formed on the safety sleeve to locate closely adjacent to one of the two lugs of the locating section and communicate with the locating slot to thereby give that lug a certain degree of elasticity.

13. The safety structure for covering a syringe needle as claimed in claim 10, further comprising two opposing protrusions provided in the locating slot between the sliding slot and the locating section; the projection on the hub being allowed to reversibly pass through the two opposing protrusions; whereby when the projection is forward moved through the two opposing protrusions to locate behind them, the safety sleeve is temporarily held to a position capable of enclosing the whole needle therein, so that the needle that has been used to draw an injection fluid but has not been used to inject is prevented from being contaminated by external environment and unexpectedly stabbing a user.

14. The safety structure for covering a syringe needle as claimed in claim 10, wherein one face of the projection on the hub facing toward the locating hole is tapered to allow the projection to smooth pass through the locating section on the safety sleeve.
